(19) United States
(12) Patent Application Publication
(10) Pub. No.: US 2024/0412828 A1
(43) Pub. Date: Dec. 12, 2024

(54) SYSTEMS AND METHODS FOR IMPLEMENTING A CLINICAL TRIAL PLATFORM

(71) Applicant: Adaptive Clinical LLC, Sarasota, FL (US)

(72) Inventors: Sina Adibi, Venice, FL (US); Arthur Isaenko, Ontario (CA)

(73) Assignee: Adaptive Clinical LLC, Sarasota, FL (US)

(21) Appl. No.: 18/732,798

(22) Filed: Jun. 4, 2024

Related U.S. Application Data
(60) Provisional application No. 63/506,451, filed on Jun. 6, 2023.

(51) Int. Cl. G16H 10/20

(52) U.S. Cl. CPC G16H 10/20

(57) ABSTRACT

A clinical trial platform includes a user interface that displays a list of source fields associated with a first third-party clinical trial tool, a list of target fields associated with a second third-party clinical data tool, and a list of transformational rules for transforming the source fields into the target fields. An artificial intelligence engine automatically suggests the source fields, target fields and transformational rules from a knowledge base of representative source and target systems. The user interface includes an editor that allows a user to overwrite one or more of the source fields, target fields or transformational rules and the knowledge base is automatically augmented in response to fields overwritten using the editor. As changes are made, the system dynamically augments the knowledge base, enabling continuous learning and adaptation to evolving data structures in clinical trials.

### CROSS-REFERENCE TO RELATED APPLICATION

This application claims the benefit of U.S. Provisional Patent Appl. 63/506,451 filed Jun. 6, 2023, the contents of which is hereby incorporated herein by reference in its entirety.

### FIELD OF THE INVENTION

This application relates generally to clinical trials and, more particularly to systems and methods for generating submissions relating to clinical trials to regulatory authorities such as the U.S. Food and Drug Administration (FDA).

### SUMMARY

A clinical trial study platform is disclosed. In accordance with one aspect, the clinical trial platform includes a user interface that displays a list of source fields associated with a first third-party clinical trial tool, a list of target fields associated with a second third-party clinical data tool, and a list of transformational rules for transforming the source fields into the target fields. An artificial intelligence (AI) engine automatically suggests the source fields, target fields and transformational rules from a knowledge base of representative source and target systems. In one embodiment, this AI engine uses machine learning and pattern recognition techniques to automatically suggest the source fields, target fields, and transformational rules, derived from a knowledge base of representative source and target systems. The user interface includes an editor that allows a user to overwrite one or more of the source fields, target fields, transformational rules and validation rules, and subsequently, any alterations are ingested by the AI engine to augment the knowledge base, promoting a continuous learning environment in response to fields overwritten using the editor. In one example, the platform automatically generates documentation in human readable form for validation based on at least an actual system configuration or mapping rules defined using the user interface. The documentation includes, for example, an FDA-compliant functional requirements specification.

In accordance with an example, a clinical trial platform includes a singular multi-tenant clinical data bus that includes a rules engine, a mapper and a plurality of bus connectors, capable of maintaining multiple disparate data-streams while maintaining strict data separation. An API gateway implements a messaging service within the components of the clinical data bus as well as to the other systems. A user interface displays a list of source fields associated with a first third-party clinical trial tool, a list of target fields associated with a second third-party clinical data tool, and a list of transformational rules for transforming the source fields into the target fields. An artificial intelligence engine automatically suggests the source fields, target fields and transformational rules from a knowledge base of representative source and target systems. The user interface includes an editor that allows a user to overwrite one or more of the source fields, target fields or transformational rules, and the knowledge base is automatically augmented in response to fields overwritten using the editor.

In certain embodiments, the user interface further includes a library for selecting data connectors and a visual editor for creating a data stream. The data connectors selected from the library are used to populate the visual editor for creating the data stream. The source fields and target fields are suggested based at least in part on the data stream created with the visual editor.

### BRIEF DESCRIPTION OF THE DRAWINGS

FIG. 1 illustrates an example of a Clinical Trial Study (CTS) Platform operable to process and map data between several categories of 3rd party software tools.

FIG. 2 is a process diagram illustrating the design flow associated with an exemplary clinical trial study.

FIGS. 3A-3C are architectural diagrams of an example of the CTS Platform.

FIG. 4 depicts an exemplary connector library used in the CTS Platform.

FIG. 5 illustrates a user interface for defining a data stream or flow using the CTS Platform.

FIG. 6 illustrates an exemplary field map list for use with the CTS Platform.

FIG. 7 depicts a user interface that allows a user to edit an automatically-generated suggested transformation rule.

FIGS. 7A-7H depict examples of documents that are automatically generated in human readable form in compliance with regulatory requirements by the CTS Platform.

FIG. 8 depicts a stream library of previously defined streams for use with the CTS Platform and user interface for configuring same.

FIGS. 9 and 10 depict examples of the auditing and monitoring operations performed by the CTS Platform.

### DETAILED DESCRIPTION

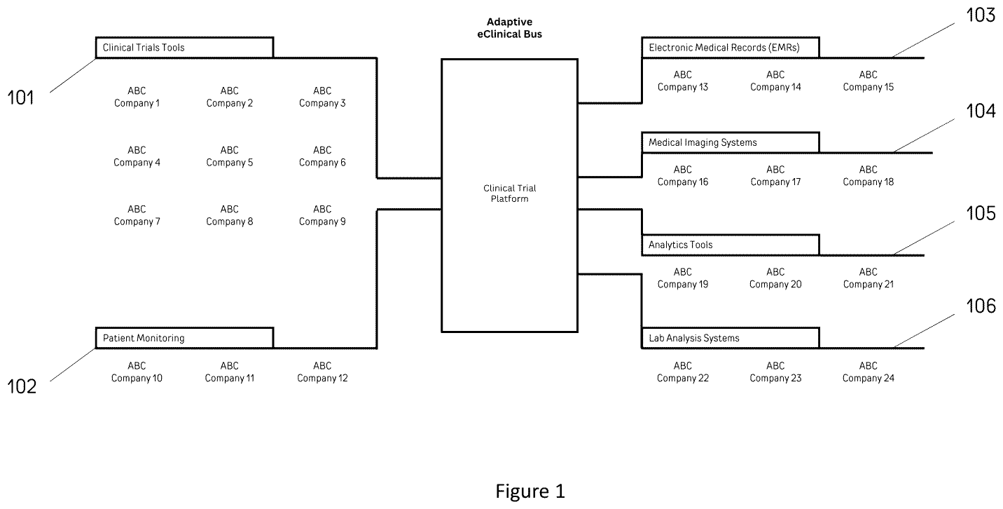

This application discloses a Clinical Trial Study Platform (CTS Platform). In one embodiment, the CTS Platform is compatible with 3rd party software tools and is operable to map data between such tools. In the example shown in FIG. 1, the CTS Platform is operable to process and map data between several categories of 3rd party software tools used in connection with clinical studies including, Clinical Trial Tools 101, Patient Monitoring Applications 102, Electronic Medical Record Tools 103, Medical Imaging System Tools 104, Analytics Tools 105 and/or Consulting and Educational Service Tools 106. Examples of Clinical Trial Tools 101 include Trial Interactive by Transperfect, Veeva Vault by Veeva and Rave EDC by Medidata; examples of Patient Monitoring Tools 102 include RAD-97 CO-Pulse Oximeter by Masimo, M+ Hub for IoT Medical Devices by Medisante, and ATCOR by CardieX; examples of Electronic Medical Records Tools 103 include Cerner Millennium® by CERNER, EPIC Suite by EPIC and athenaOne by AthenaHealth>; examples of Medical Imaging System Tools 104 include Mint Lesion by Mint Medical GmbH, Myrian by Intrasense and Intelemage by Medidata Imaging; examples of Analytics Tools 105 include SPSS by IBM, Spotfire by TIBCO and Clinical Trial Intelligence Platform by Lokavant; and examples of Consulting and Educational Service Tools 106 include Blue Sky eLearning by Blue Sky and CitiusTech Services by CitiusTech.

The CTS Platform reads data from the third-party data sources using “connectors.” Using a registry of connectors, the CTS Platform can access any of the data sources shown, for example, in FIG. 1. In one example, the CTS Platform uses artificial intelligence to generate rules (“AI Rules”) for mapping data between the third party tools. The AI rules are generated both statically (from specifications) or dynamically—by inferring them from pre-existing files, specifications, database schemas or other sources. For example: “Date of Birth” format requirements are often specified in the protocol and thus are statically transformed. In this respect, the protocol is the statistical definition document that is reviewed and approved by the FDA that specifies the type and the manner with which data is collected from patients for a given clinical trial. Alternatively, in the case of “Pregnancy Test Results” a dynamically generated rule will only display if, for example, gender is determined to be female. The CTS Platform also includes a “Mapper system” with a Mapper UI, Mapper API, Mapper Processor and Mapper Rules. Once the Mapper system creates the AI Rules, then the rules are used by the Rules Engine which is integrated into a Clinical Data Bus to dynamically transform the data from the source to the destination.

In one embodiment, the CTS platform includes several features including: (i) an artificial intelligence model that automates data modeling using input and output sample files, templates or scripts, (ii) a Connector Library capable of deploying data readers/data writers according to third-party data input/output requirements, (iii) an online mapper that leverages the AI Rules to initialize and automatically suggest mappings while allowing user control and adjustments, (iv) a Clinical Rules Engine capable of data aggregation and transformation based on predefined and user defined clinical rules, (v) a Process Control Module enabling definition, activation, and deactivation of data streams, (vi) a Visualization Module for ongoing monitoring of data streams with search functions, filters, alerts and operator decision support tools based on data driven/AI created flags, as well as rule based flags and user defined flags, and (vii) a Self-service Interface that enables users to define data streams for clinical trial data processing and integration.

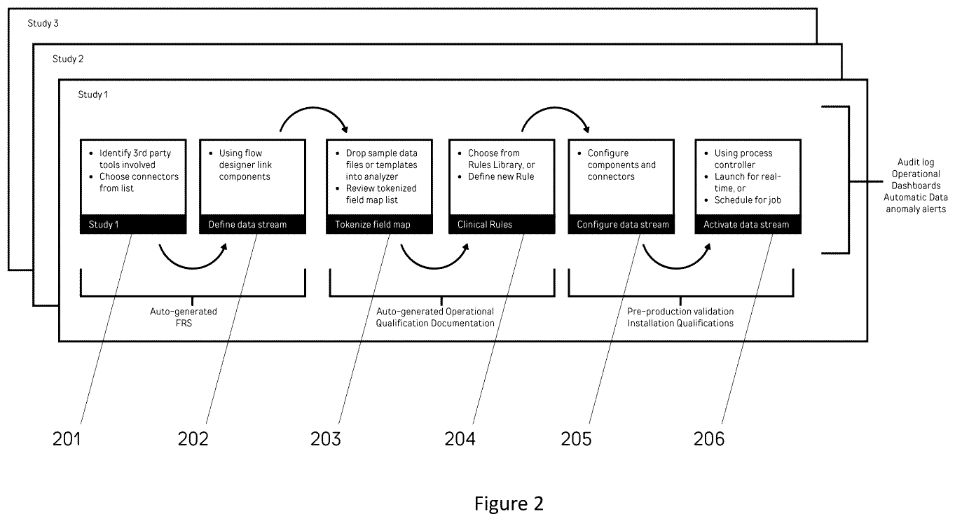

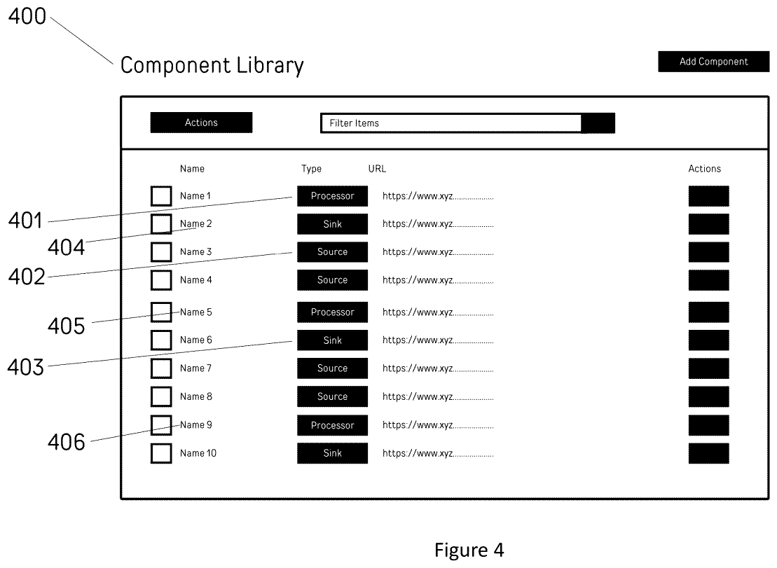

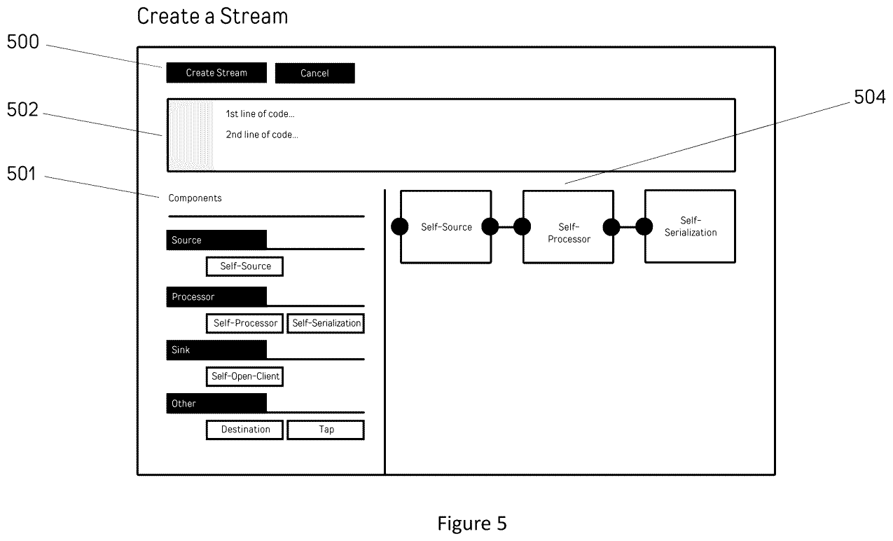

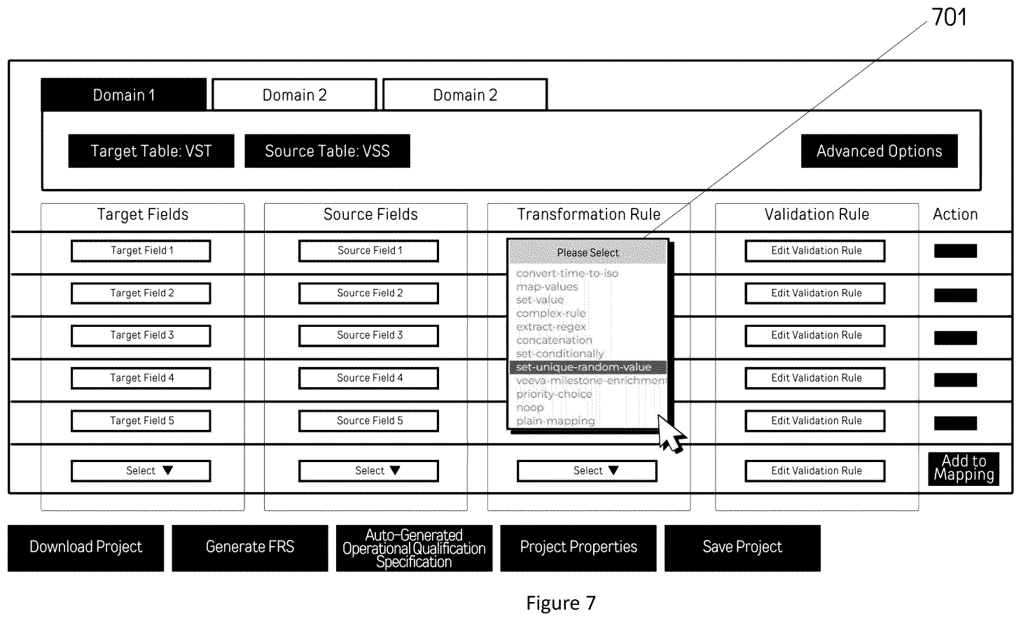

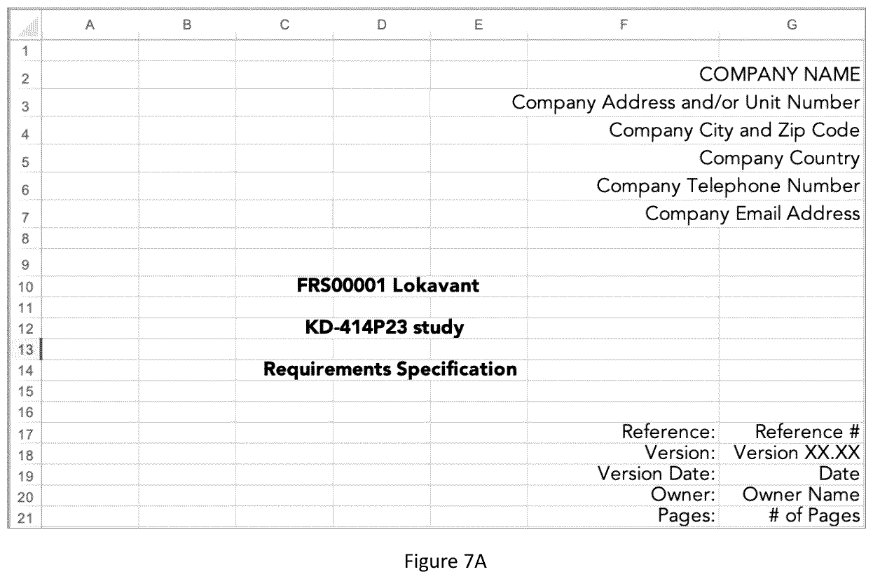

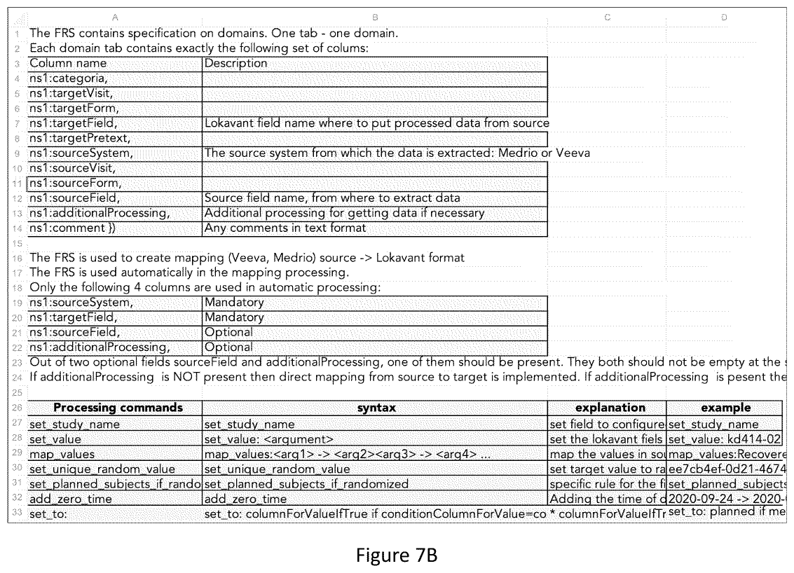

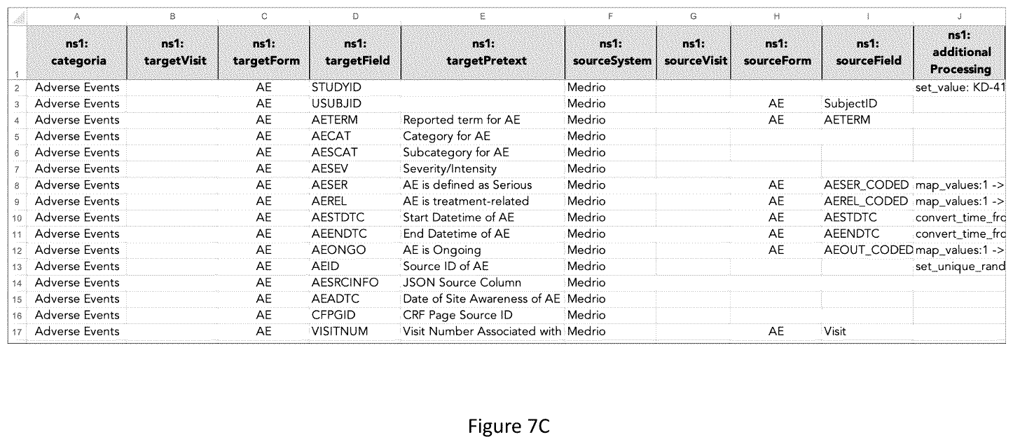

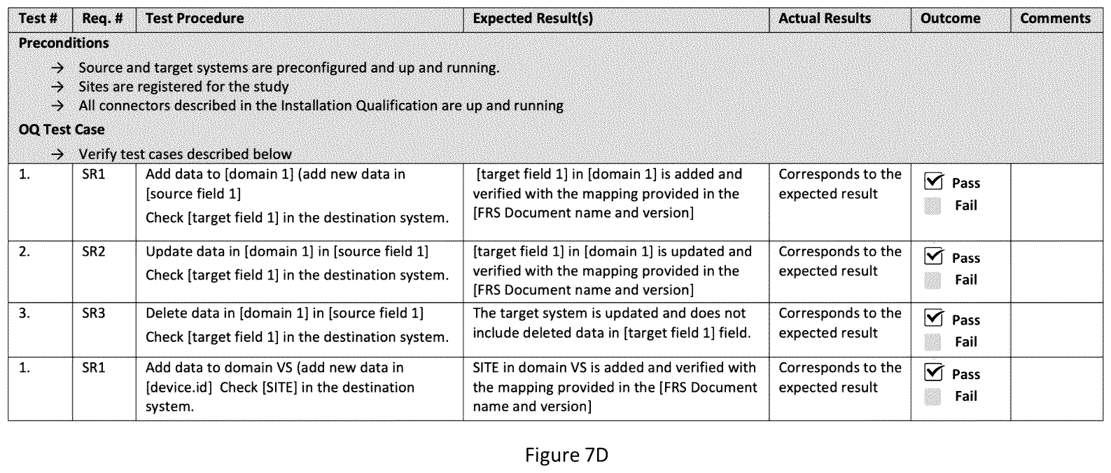

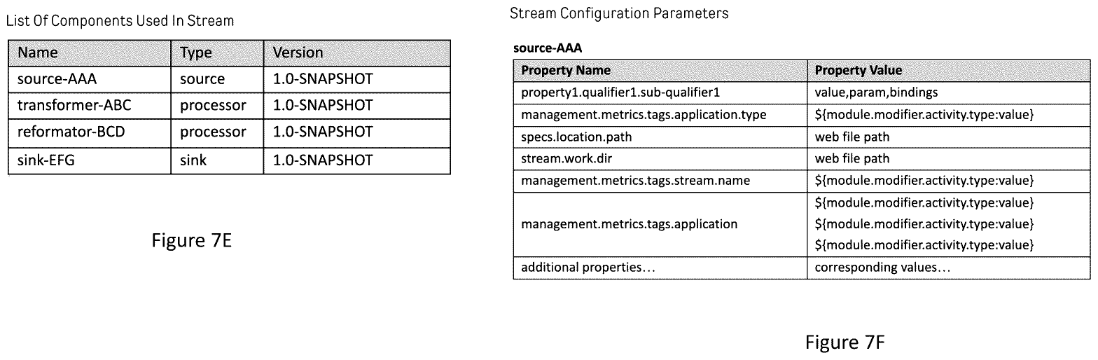

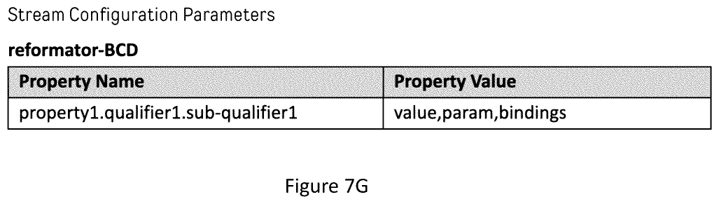

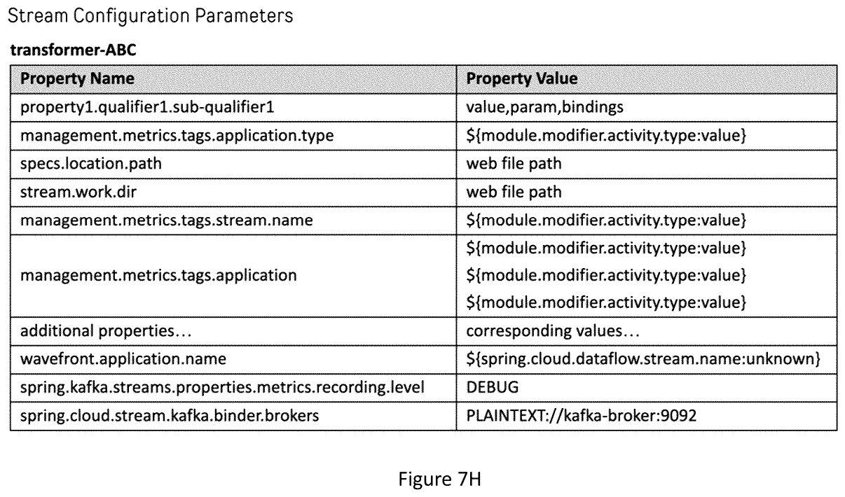

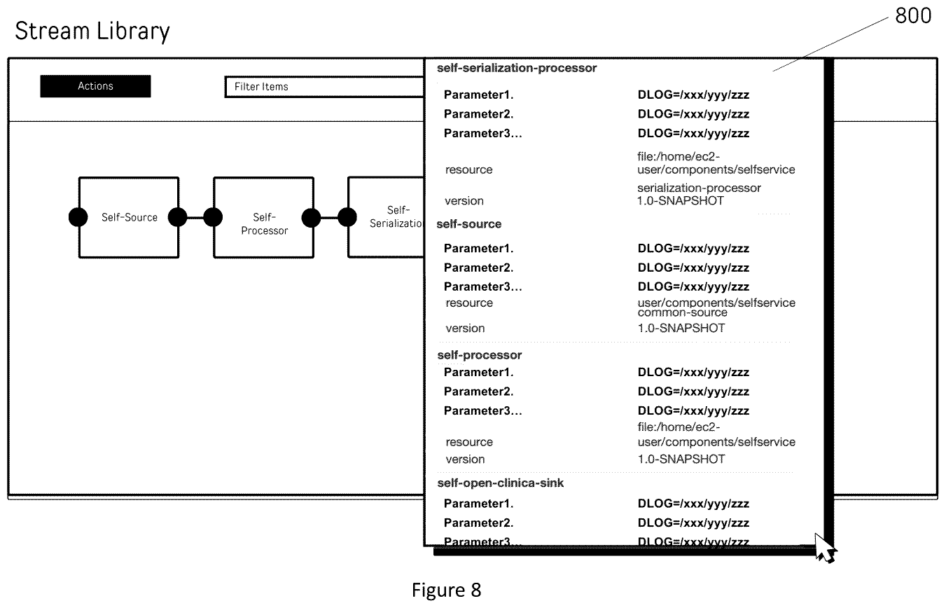

FIG. 2 is a process diagram illustrating the design flow associated with an exemplary clinical trial study. In step 201, a study setup process is performed. In this step, the user selects the third-party software tools applicable to the study, and chooses connectors from a connector library 400 for mapping data between the third-party tools. An example of connector library 400 is shown in FIG. 4. In step 202, the user defines a data stream or flow using designed link components. An exemplary interface for defining a data stream or flow is shown in FIG. 5 (described below). Steps 201 and 202 automatically generate a functional requirements specification (FRS) for submission to the U.S. Food and Drug Administration (FDA). An example of an FRS is shown in FIGS. 7 and 8. While the disclosed embodiment is described in connection with submissions to the FDA, it will be understood that the present disclosure is applicable to submissions to other regulatory bodies including, for example, submissions to the European Medicines Agency (EMA), the Pharmaceuticals and Medical device Agency in Japan and/or the National Medical Products Administration in China.

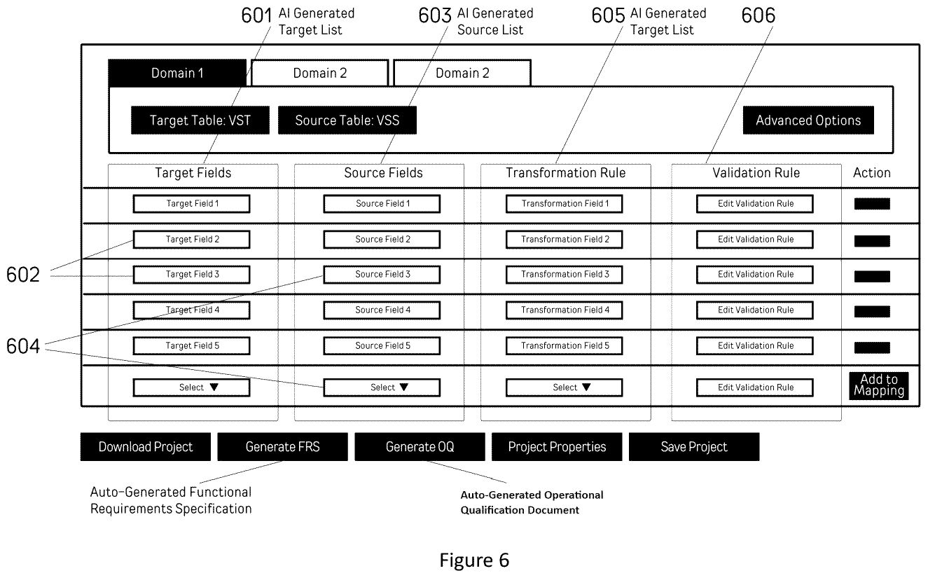

Referring still to FIG. 2, in step 203, the user identifies sample data files or templates from the applicable third-party tools. Using the AI Rules, the CTS Platform generates a tokenized field map list. As exemplary field map list is shown in FIG. 6, and includes a column with target fields and a column with corresponding source fields. FIG. 6 will be discussed in further detail below. In step 204, the user selects (from a Rules Library) or defines a rule for mapping each target field with its corresponding source field. Steps 203 and 204 automatically generate validation scripts compliant with 21 CFR Part 11, that when executed, are ready for submission to the FDA. In step 205, the user configures the components and connectors by providing server parameters such as 3rd party server location, memory allocation, scratch directories, cluster counts, etc., and in step 206 the user activates the stream. As part of step 206, the CTS Platform produces audit logs and provides data anomaly alerts via a dashboard screen.

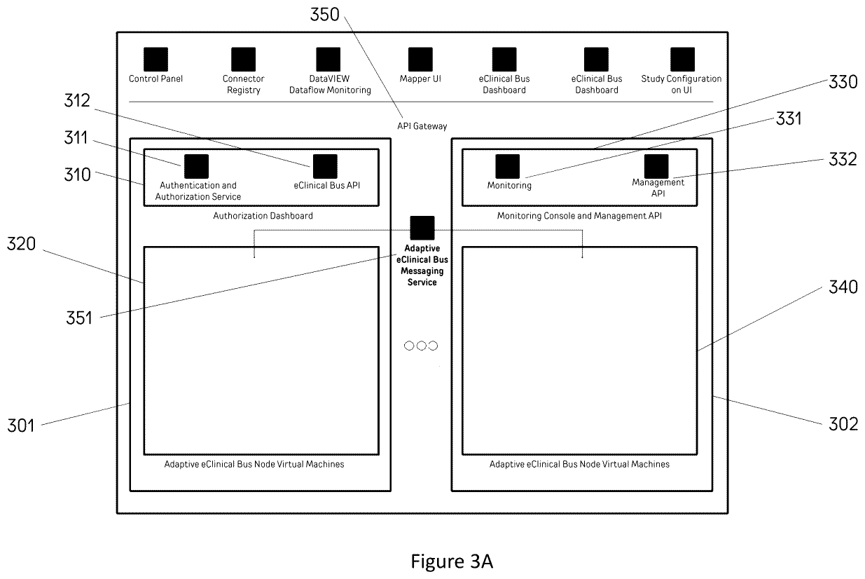

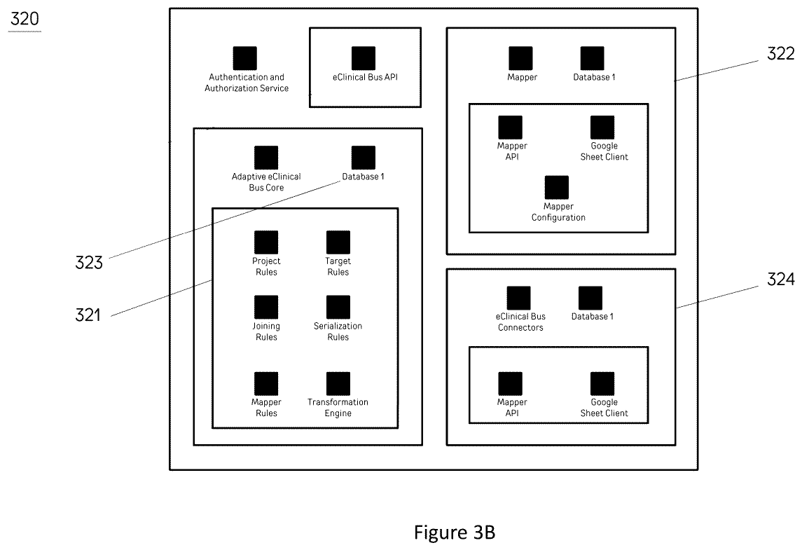

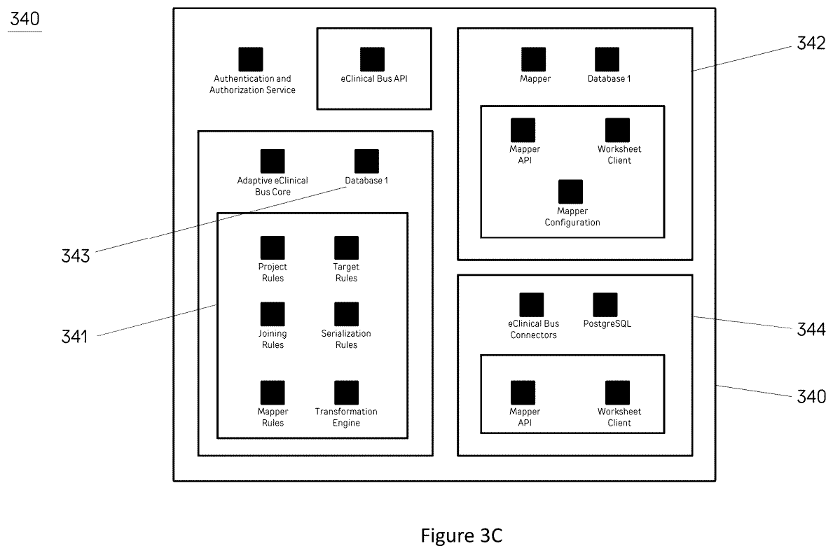

FIGS. 3A-3C are an architectural diagram of the CTS Platform. Functional component 301 includes a Dashboard module 310 and a first instance of a Clinical Data Bus node or virtual machine 320 (FIG. 3B). Functional component 302 includes a Monitoring Console and Management API module 330 and a second instance of a Clinical Data Bus node or virtual machine 340 (FIG. 3C), if configured as such. While in the diagram shown, two nodes (or virtual machines) are shown, it will be understood that more of fewer or such virtual machines can be used. In the example shown, functional components 301 and 302 are coupled over an API Gateway 350 that implements a Clinical Data Bus messaging service 351. Clinical Data Bus modules 320, 340 include a Rules Engines 321, 341, respectively. Each Rule Engine 321, 341 includes several categories of rules including project rules, target rules, joining rules, serialization rules and mapper rules, as well as a serialization engine. Project rules govern the overall controls of all other rules within a given project. Target, source, joining and serialization rules are embedded within the project and further qualify the behavior of the data stream as it relates to the 3rd party source and/or target system, manner of defining the inter-relationship of data elements (joining) and transformation of the resultant structured data into a flat data sequence that is consumable by the target system (serialization). Clinical Data Bus nodes 320, 340 also include (i) mappers 322, 342, (ii) data warehouses 323, 343, and (iii) bus connector modules 324, 344, in each case, respectively. Alternatively, a singular multi-tenant clinical data bus is used that includes a rules engine, a mapper and a plurality of bus connectors, capable of maintaining multiple disparate data-streams while maintaining strict data separation. Dashboard module 310 includes an authentication and authorization service 311 and a Clinical Bus API 312. Monitoring Console and Management API module 330 includes Monitoring Service 331 and a Management API 332.

In one embodiment, the architecture of the CTS Platform includes controls and safeguards for compliance with FDA and HIPAA security requirements and integrity management regulations including ALCOA (Attributable, Legible, Contemporaneous, Original and Accurate) requirements. In one example, the CTS Platform automatically generates an FDA compliant Installation Qualification based on the actual system configuration, and an FDA compliant Operational Qualification based on the defined mapping rules and data stream configuration. The modularized architecture enables elemental testing, verification and validation according to FDA regulations.

Referring now to FIG. 4, a connector library 400 is shown for mapping data between the third-party tools. Connector library 400 includes multiple connector types, including a processor type 401, source type 402, and a sink type 403. Each source type connector 402 corresponds to a specific version of a 3rd party tool (example: Argus Medical System) that is providing data into the data stream using its preferred method such as public/private, standards based or proprietary means of reading data from same. Each processor type connector 401 corresponds to modules that run the instructions generated using the mapping wizard as well as other manually entered instruction sets. There can be many processor types in any given data stream as needed as they are components that do the majority of the work. Each sink type connector 403 corresponds to a specific version of a 3rd party tool that is receiving data from the data stream using its public/private, standards based or proprietary means of reading data from same. The connectors are adapted to interface with the third-party tools shown on FIG. 1. For example, connector 404 may be adapted to interface with the Bioclinica Clinical Trial tool, and connector 405 is adapted to interface with the Medrio Clinical Trial tool. The user can select various connectors from the library by clicking a checkbox, such as checkbox 406. In one embodiment, library connectors are only visible to users according to their username and permissions. The connector library feature simplifies and speeds up implementation of the CTS Platform for users, eliminating the need for specialized support from CTS Platform staff.

FIG. 5 illustrates a user interface 500 for defining a data stream or flow. Connectors selected from connector library 400 are populated in a visual form into components section 501, and in text form in window 502. A user creates a stream by ordering and linking the connectors within area 504. In one example, user interface 500 is configured with guardrails to only allow on a subset of modules to be selected from connector library 400 in order to simplify the platform for new users, and to avoid errors in connections. Once a stream is defined, it can be stored in the stream library 800 (shown in FIG. 8) for reuse whereupon revised run-time parameters are specified according to that particular instance of the data stream.

FIG. 6 illustrates an exemplary field map list which includes target field column 601 with target fields 602 and source field column 603 with corresponding source fields 604. Source field column 603 is generated by performing an AI-driven analysis on an exemplary file or database associated with the applicable third-party source tool. Similarly, target field column 601 is generated by performing an AI-driven analysis on an exemplary file or database associated with the applicable third-party target tool. Alternatively, the entries in source field column 603 and target field column 601 are generated manually. In an example where source field column 603 and/or target field column 601 is initially populated using an AI-driven analysis, source fields 604 and/or target fields 602 can be overwritten by the user. When a source field 604 or target field 602 is overwritten by the user, the result is automatically added to the AI knowledge base. Subsequently, any alterations are ingested by the AI engine to augment the knowledge base, promoting a continuous learning environment.

Referring still to FIG. 6, the exemplary field map list includes a transformation rule column 605 and a validation rule column 606. For each row in FIG. 6, the entry in column 605 corresponds to a rule or rule combinations for transforming corresponding source field 604 into the corresponding target field 602. In one example, the entries in transformation rule column 605 are automatically suggested by the AI model based on a knowledge base of representative source and target systems. The user is then allowed to edit the automatically-generated suggested transformation rule using, for example, menu 701 (shown in FIG. 7). Again, when a suggested transformation rule is overridden by the user, the result is automatically added to the AI knowledge base. For each row in FIG. 6, the entry in column 606 corresponds to a rule or rule combinations for validating source field 604 prior to transformation of the information into the corresponding target field 602. In one example, the entries in validation rule column 606 are input by a user and/or automatically suggested by the AI model based on a knowledge base of representative source information. An exemplary validation rule checks, e.g., whether information in the source field is within an allowable range. The user is allowed to edit the validation rules, including those automatically-generated and suggested, using a menu (not shown). Again, when a suggested validation rule is overridden by the user, the result is automatically added to the AI knowledge base. Once the entries in columns 601, 603, 605 and 606 are finalized and saved, several output screens (shown in FIGS. 7A-7H) are in automatically generated in human readable form in compliance with regulatory requirements. In the example shown, these output screens provide an FDA compliant functional requirements specification on the actual system configuration, as well as the defined mapping rules and data stream configuration.

The features of the user interface shown in FIGS. 6 and 7 allow users to gain from the intelligence built into the CTS Platform to perform tasks that otherwise would require a deep understanding of source and target data structures and data exchange standards. The auto-suggest functionality built into the interface eliminates time consuming repeated manual mapping operations and significantly reduces analysis and discovery efforts. In addition, the level of technical sophistication of the user is greatly reduced thus enabling otherwise inexperienced users to perform tasks that would otherwise need to be performed by more highly skilled technical and experienced workers.

As shown in FIGS. 7A-7H, FDA-compliant documentation necessary for validation of key system components is automatically generated in human readable form by the CTS Platform. In conventional clinical data management systems, staff with training in FDA Quality Management as well as highly skilled technical data scientist are responsible for creation of such documents. This requires a unique (and difficult to find) combination skill set that includes an understanding of both technical and regulatory issues. The CTS Platform eliminates the need for such specialized skilled staff by automatically generating the documentation in a form that is ready for inspection, execution and filing. Users are able to adjust the automatically-generated documentation using common tools such as Microsoft Office to insert their own additional information such as user profiles and training backgrounds. In one example, the automatically-generated FDA-compliant documentation includes a complete FDA compliant Functional Requirements Specification, an Operational Qualification Document and an Installation Qualification Document. FIG. 7A depicts the cover of such a requirements specification. FIG. 7B depicts a typical description of data domains that are within scope of said document FIG. 7C depicts page(s) addressing target forms, fields and edit checks if applicable FIG. 7D depicts page(s) of validation steps as required during Operational Qualification of the system as derived automatically from the overall system parameters and the functional requirements specification as in FIGS. 7A, 7B and 7C. FIGS. 7E, 7F, 7G and 7H depict page(s) of validation steps required during Installation Qualification of the system as automatically derived from the overall system parameters.

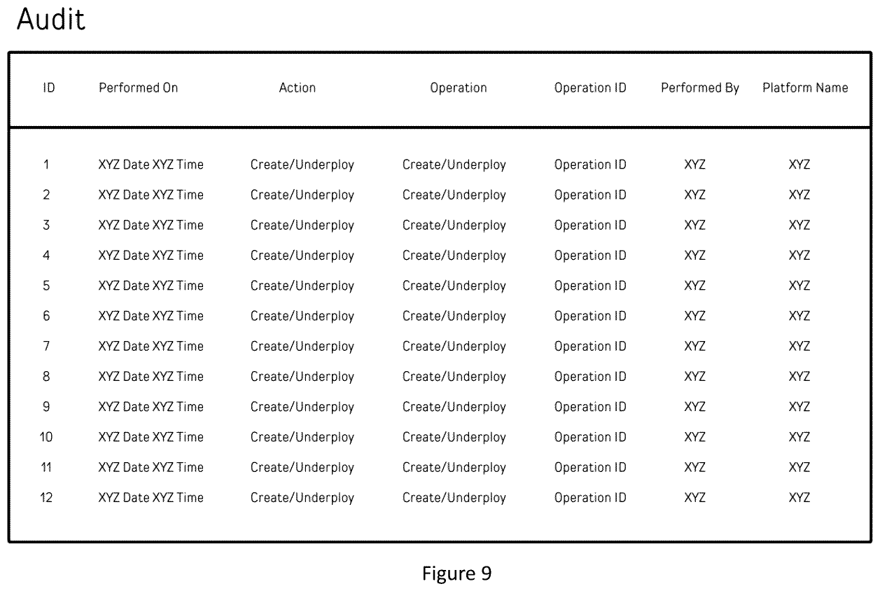

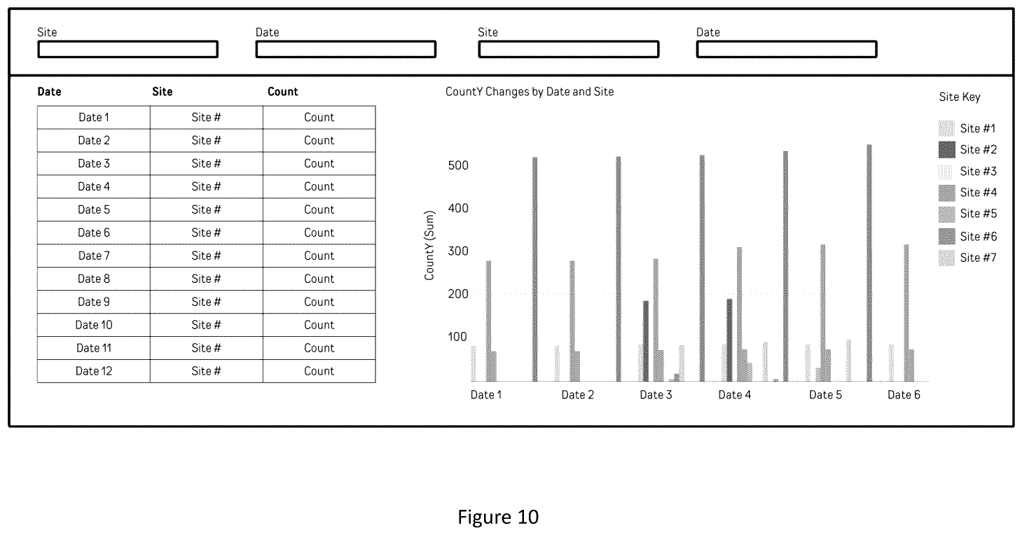

FIGS. 9 and 10 depict examples of the auditing and monitoring operations performed by the CTS Platform. FIG. 9 depicts an audit log maintained by the CTS Platform of each action performed on the platform. FIG. 10 is a report depicting operational metrics associated with use of the CTS Platform. FDA regulations require any system involved with patient or research data to comply with ALCOA requirements. This requires that the operator(s) of any such system be able to assure accuracy of data and that it is attributable. When combining data streams from disparate sources the challenges associated with these requirements become exponentially more difficult because disparate activities need to be combined, presented, monitored and reported together.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A clinical trial platform, comprising:
a user interface that displays a list of source fields associated with a first third-party clinical trial tool, a list of target fields associated with a second third-party clinical data tool, and a list of transformational rules for transforming the source fields into the target fields;
a knowledge base of representative source and target systems;
wherein the user interface automatically suggests the source fields, target fields and transformational rules from the knowledge base of representative source and target systems;
wherein the user interface includes an editor that allows a user to overwrite one or more of the source fields, target fields or transformational rules, and wherein the knowledge base is automatically augmented in response to fields overwritten using the editor; and
wherein the platform automatically generates documentation in human readable form for validation based on at least an actual system configuration or mapping rules defined using the user interface.

2. The clinical trial platform of claim 1, wherein the documentation comprises an FDA-compliant functional requirements specification.

3. The clinical trial platform of claim 1, wherein the user interface a list of validation rules for validating the source fields and data into the target fields; and
wherein the user interface includes an editor that allows a user to overwrite one or more of the validation rules, and wherein the knowledge base is automatically augmented in response to validation rules overwritten using the editor.

4. A clinical trial platform, comprising:
a first data bus node that includes a mapper and a plurality of bus connectors;
a second data bus node that includes a mapper and a plurality of bus connectors;
an API Gateway that implements a messaging service between the first data bus node and the second data bus node;
a user interface that displays a list of source fields associated with a first third-party clinical trial tool, a list of target fields associated with a second third-party clinical data tool, and a list of transformational rules for transforming the source fields into the target fields;
wherein the user interface automatically suggests the source fields, target fields and transformational rules from a knowledge base of representative source and target systems;
wherein the user interface includes an editor that allows a user to overwrite one or more of the source fields, target fields or transformational rules; and
wherein the knowledge base is automatically augmented in response to fields overwritten using the editor.

5. The clinical trial platform of claim 4, wherein the user interface a list of validation rules for validating the source fields and data into the target fields; and
wherein the user interface includes an editor that allows a user to overwrite one or more of the validation rules, and wherein the knowledge base is automatically augmented in response to validation rules overwritten using the editor.

6. The clinical trial platform of claim 4, wherein the user interface further comprises a library for selecting data connectors and a visual editor for creating a data stream.

7. The clinical trial platform of the claim 6, wherein data connectors selected from the library are used to populate the visual editor for creating the data stream.

8. The clinical trial platform of claim 7, wherein the source fields and target fields are suggested based at least in part on the data stream created with the visual editor.

9. The clinical trial platform of claim 8, wherein the platform manages activation and deactivation of data streams.

10. The clinical trial platform of claim 9, wherein the platform provides for real-time monitoring of data streams.

11. The clinical trial platform of claim 10, wherein the platform further comprises a self-service interface enabling users to define data streams for clinical trial data processing and integration.

12. A clinical trial platform, comprising:
a processor; and
a memory storing instructions that, when executed by the processor, cause the processor to perform operations including:
displaying a user interface that includes a list of source fields associated with a first third-party clinical trial tool, a list of target fields associated with a second third-party clinical data tool, and a list of transformational rules for transforming the source fields into the target fields;
automatically suggesting, via the user interface, the source fields, target fields and transformational rules from a knowledge base of representative source and target systems; wherein the user interface includes an editor that allows a user to overwrite one or more of the source fields, target fields or transformational rules;
automatically augmenting the knowledge base in response to fields overwritten using the editor; and
automatically generating documentation in human readable form for validation based on at least an actual system configuration or mapping rules defined using the user interface.

13. The clinical trial platform of claim 12, wherein the user interface a list of validation rules for validating the source fields and data into the target fields; and
wherein the user interface includes an editor that allows a user to overwrite one or more of the validation rules, and wherein the knowledge base is automatically augmented in response to validation rules overwritten using the editor.

14. The clinical trial platform of claim 12, wherein the documentation comprises an FDA-compliant functional requirements specification.

15. The clinical trial platform of claim 12, wherein the instructions, when executed by the processor, further cause the processor to operate a first data bus that includes a mapper and a plurality of bus connectors, a second data bus that includes a mapper and a plurality of bus connectors, and an API Gateway that implements a messaging service between the first data bus and the second data bus.

16. The clinical trial platform of claim 12, wherein the user interface further comprises a library for selecting data connectors and a visual editor for creating a data stream.

17. The clinical trial platform of the claim 16, wherein data connectors selected from the library are used to populate the visual editor for creating the data stream.

18. The clinical trial platform of claim 17, wherein the source fields and target fields are suggested based at least in part on the data stream created with the visual editor.

19. The clinical trial platform of claim 18, wherein the platform manages activation and deactivation of data streams.

20. The clinical trial platform of claim 12, wherein the user interface further comprises a self-service interface enabling users to define data streams for clinical trial data processing and integration.
